(19) United States
(12) Patent Application Publication
(10) Pub. No.: US 2007/0179523 A1
(43) Pub. Date: Aug. 2, 2007

(54) Self acupressure device

(76) Inventor: William Jing, San Carlos, CA (US)

Correspondence Address: William Jing, 1185 Laurel Street, San Carlos, CA, 94070, US

(21) Appl. No.: 11/344,308

(22) Filed: Jan. 31, 2006

(51) Int. Cl. A61B 17/00

(52) U.S. Cl. 606/204

(57) ABSTRACT

The invention relates to a self-acupressure device and method for alleviating back strains. The device comprises an elongated flexible casing containing one or more acupressure elements at one or both ends of the elongated flexible casing By swinging the acupressure elements horizontally from side to side or diagonally over the shoulder and under the armpit of a subject with the application of different speeds and force, the subject can apply acupressure stimulations to the selected areas of his/her own back by hitting the selected areas of the body with the selected intensity and frequency of the hits. The elongated casing can be used to extend the hands of a subject. Soft acupressure elements can be used for imitating acupressure massage performed by the palm a human hand, and hard acupressure elements for imitating acupressure massage performed by the thumb of a human hand.

### CROSS-REFERENCE TO RELATED APPLICATIONS

This application is based on provisional patent application Ser. No. 60/628,463 filed Nov. 16, 2004.

### FIELD OF THE INVENTION

The invention relates to devices and methods involved in the therapeutic application of acupressure to a subject in need thereof, in particular, to his or her back.

### BACKGROUND OF THE INVENTION

Back strains are common in the computerized world. People sit in front of computers daily for work, education, and even recreation with strenuous postures. Back strains are daily phenomena among office employees, scientists, and even among students. Back strains cause inconvenience and suffering.

The treatment for back strains is costly and time consuming. A lot of people go to acupressure or massage therapists at the early stage of their back discomfort. Accumulation of back strains jeopardizes their health and leads them to doctors' visits. Back strain often results in absenteeism from work and high cost of medical bills.

Back strains cannot be cured easily by health professionals. Back strain patients cannot help themselves either. To do acupressure massage on his/her own back is difficult because there are places on the back of a subject that his/her hands can hardly reach and manipulate. Most often a subject has to bear the pain and discomfort. Back strains may accumulate and cause serious health problems in the long run.

Finding methods and tools for effective alleviation of the back strains has attracted inventors' attention since long ago. A lot of attempts have been made to perform acupressure and massage for improving blood circulation of the strained muscles in the back.

For example, U.S. Pat. No. 5,848,980 issued to Demerais in 1998 describes a hoop-shaped massage apparatus that can be fitted with a vibrator massage unit. The hoop is positioned around a selected body area of a user to apply pressure to the user's body. However, the apparatus is bulky and inconvenient to use or transport.

U.S. Pat. No. 4,266,536 issued to Casares in 1981 describes a back massager reported to be capable of massaging the user's back without assistance. The massager has a curved shaft having a handle near the first end and a second handle positioned at an intermediate point between the first and second ends of the shaft. The shaft is curved and has roller means affixed at the second end thereof. However, the device is awkward to use and bulky. There is no practical way for a subject to use the device to apply acupressure to his or her own back.

U.S. Pat. No. 6,010,467 issued to Smith in 2000 describes an apparatus reportedly enabling the patient himself or herself to engage the skin on his or her back to locate and treat trigger points thereunder, using a source of pulsed voltage. The apparatus includes a wand having a generally rigid and electrically-conductive arcuate handle of about an arm's length or less in length, a spade-shaped electrically-conductive extension with a free end formed with tapering straight edges merging in a rounded point and welded on its other end to the handle, and a cable secured on the other end of the handle to connect the extension electrically to the source of pulsed voltage. A grip at the other end of the handle facilitates moving the wand so that a tapering straight edge and/or the rounded point of the spade-shaped extension glides over the back's skin when searching for trigger points, and for holding the rounded point there when a trigger point is located to enable precise electrically stimulation of the tissue near the trigger point. However, the apparatus is designed to electrically stimulate “trigger points,” which is not an intended effect of acupressure.

U.S. Pat. No. 4,479,495 issued to Isaacson in 1984 describes an acupressure device for applying pressure to specific points on a human body comprising a stimulator which is attached to a flexible cinching strap or band to extend circumferentially around a body part, including a releasable connecting means to facilitate the positioning and securing of the terminus of the stimulator against a specific point on the human body. The pointed surface end can be made to contact and apply pressure to a selected point on the user's body by extending the strap around a portion of the body and tightening same with the terminus of the stimulator in proper position. However, this device can apply only a static pressure that is not capable of repetitive or rhythmic application. In addition, the device cannot be used to apply variable pressure to one or more acupressure points in rapid succession.

U.S. Pat. No. 5,938,684 issued to Lynch, et al in 1999 describes an appliance for therapeutic pressure application comprising at least one protuberance each having a base end anchored to a resilient medium and a point end for applying pressure to tissue, the medium having resilient, flexible and insulating properties to absorb in part the pressure between the point ends and the tissue so as to prevent pain, cut or penetration of the tissue by the point ends and to retain the heat generated by the tissue. However, this device can apply only a static pressure that is not capable of repetitive or rhythmic application. In addition, the device cannot be used to apply variable pressure to one or more acupressure points in rapid succession.

U.S. Patent Application Publication No. 2005/021598 filed by Banks, et al. and published in 2005 discloses the use if a flexible roller that applies pressure parallel and adjacent to the spine and along the length of the roller. The acupressure is performed by a patient who carries out rocking motions over the roller that is used as a fulcrum.

However, the products described above and other products for self-treatment known to the inventor herein do not really satisfy a back strain patient to full extent. The major problems are the inconvenience to use and difficulty to control. It is hard for the patient to use a prior art product to give himself/herself acupressure on the selected spots of his/her back and move the equipment back and forth and/or side-to-side with full control of the frequency and hardness of the stimulations he wishes. Moreover, the known devices are heavy and bulky, not really portable, so the patient cannot use them to find relief from back strains when traveling or at work. Many of the devices are costly to manufacture and too complicated to operate.

Therefore, a need exists for a really portable and easy to operate device to provide acupressure for back strains, and that device should enable a patient to do acupressure massage on the selected areas of the body, e.g., on the back, with a good control of the intensity of the acupressure massage by himself/herself without the assistance of others. And ideally the device should be convenient in use and inexpensive in the manufacture.

### SUMMARY AND OBJECTS OF THE INVENTION

It is an object of the invention to provide a self-acupressure device for self-acupressure that is easy to use, readily portable and inexpensive to manufacture.

It is another object of the invention to provide a device that enables the subject to give acupressure to the selected areas, especially on his/her back.

It is a further object of the invention to provide a device that allows acupressure stimulation with the intensity and frequency as the subject prefers.

It is an object of the invention to provide a device for acupressure without much effort from the user in the absence of electrical power.

It is an object of the invention to provide a device that enables the subject to select and change the intensity and frequency of acupressure stimulations instantly.

It is another object of the invention to provide a method of self-acupressure treatment that is simple and can be performed in any convenient place without the use of bulky equipment.

Accordingly, a self-acupressure device is provided, comprising a hollow elongated flexible casing for containing at least one acupressure element that can be positioned at one end of the hollow elongated flexible casing. A application method is also provided, comprising swinging at least one acupressure element in the hollow elongated casing horizontally side to side and/or diagonally over the shoulder and under the armpit of a subject to strike his/her selected areas in the back.

In a preferred embodiment, two different acupressure elements are placed in a hollow elongated flexible casing, which is used to extend the hands of a subject, especially for the hard to reach areas in the back of the subject. One of the acupressure elements is soft, imitating the palm of a human hand for applying light pressure; the other acupressure element is medium hard, imitating the thumb of a human hand for applying harder pressure. A subject can choose either one of the acupressure elements at a time. When a subject needs soft acupressure on the selected areas of his/her strained back, he/she can choose and swing the soft acupressure element to strike the selected areas in his/her back. When a subject needs harder acupressure, he can choose and swing the harder acupressure element to make contact with the selected area in his/her back. A subject can also choose two acupressure elements and swing two acupressure elements jointly at the same time. The two-acupressure elements can be positioned at the two ends of a hollow elongated flexible casing. A subject can fold the elongated flexible casing by grasping it in the middle and swing the two acupressure elements positioned at the two parallel branches of the folded flexible casing to make contact with a larger selected area in his/her back with two close hits of the two elements. A subject can also shift the two acupressure elements into one end of the elongated flexible casing and wrap the empty end of the casing around one of the subject's hands and swing the two acupressure elements positioned at one end of the hollow elongated flexible casing for deep acupressure on a larger selected areas in his/her back with a joint hit of two balls. Furthermore, there is a whole range of acupressure impacts available for the subject when he/she chooses and swings the soft ball, the hard ball, the two-joint-balls at two ends, or the two-joint-balls at one end respectively. The subject can get various degrees of acupressure impacts to meet his/her needs by changing and alternating his swinging speed and firmness in slower/faster and/or soft/harder motions he/she chooses.

Two kinds of preferred acupressure elements are soft and medium hard balls, which are about 5 to 8 cm in diameter, where the soft balls may weight about 30 to 35 grams, the hard balls about 36 to 42 grams. In different embodiments, the diameters of the balls can vary from 2 cm to 30 cm and their weights vary accordingly. The acupressure element is not necessarily in the form of a ball and may be made in the form of resilient cylindrical or oval bodies. The acupressure element can be a uniform structure (e.g., 100% polyurethane foam), or may have a composite structure (e.g., in the form of a polyester bag filled with cotton fibers or seeds). Preferably, the acupressure element is in a substantially round, spherical or oval shape. The acupressure element is preferably deformable upon making contact with the subject's back or other parts of the body so that it absorbs sufficient energy of impact that the subject does not become bruised or injured, while the contact is strong enough to help the blood circulation. An acupressure element can also be formed as a soft closed container filled with a bulky material such as small particles of polyester, polyurethane, plant seeds, or the like.

In order to make the operation repetitive and accurate each time to contact the selected point on the patient's body, the casing should be made from a non-stretchable fabric, such as satin, silk. The elongated flexible casing can comprise a woven material or a nonwoven material. Preferably, the woven material is selected from cloth, netting, or mesh, or the like, which can be formed from conventional fibers, such as cotton, polyester, and so forth. The intermediate part of the casing can be made in the form of a cord or a belt.

### BRIEF DESCRIPTION OF THE DRAWINGS

FIG. 1 illustrates an embodiment of the acupressure device of the invention with the use of one acupressure element at one end of the elongated flexible element.

FIG. 2 illustrates a second embodiment of the acupressure device of the invention with the use of two acupressure elements at opposite ends of the elongated flexible element.

FIG. 3 illustrates the second embodiment of the acupressure device of the invention with the use of two acupressure elements at one end the elongated flexible element.

FIG. 4 illustrates a third embodiment of the acupressure device of the invention with the use of a cord connected to one elongated flexible element that contains one acupressure element.

FIG. 5 illustrates a fourth embodiment of the acupressure device of the invention that consists of two elongated flexible elements connected by a cord, each flexible element containing an acupressure element.

FIGS. 6 (A) through (D) show examples of various shapes and forms of the acupressure elements suitable for the invention.

FIGS. 7 to 10 illustrates various ways of using the self-acupressure devices by a patient who holds the flexible element by one hand (FIG. 7), two hands (FIG. 9), with the use of one (FIG. 7) or two (FIG. 10) acupressure elements, and by performing diagonal (FIG. 8) or horizontal (FIG. 10) swinging motions.

### DETAILED DESCRIPTION OF THE INVENTION

Before the present invention is described in detail, it is to be understood that unless otherwise indicated this invention is not limited to specific materials, dimensions, masses, or the like, as such may vary. It is also to be understood that the terminology used herein is for the purpose of describing particular embodiments only and is not intended to limit the scope of the present invention.

It must be noted that as used herein and in the claims, the singular forms “a,” “and” and “the” include plural referents unless the context clearly dictates otherwise. Thus, for example, reference to “an acupressure element” includes two or more acupressure elements; reference to “an opening” includes two or more openings, and so forth.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Self-Acupressure Devices

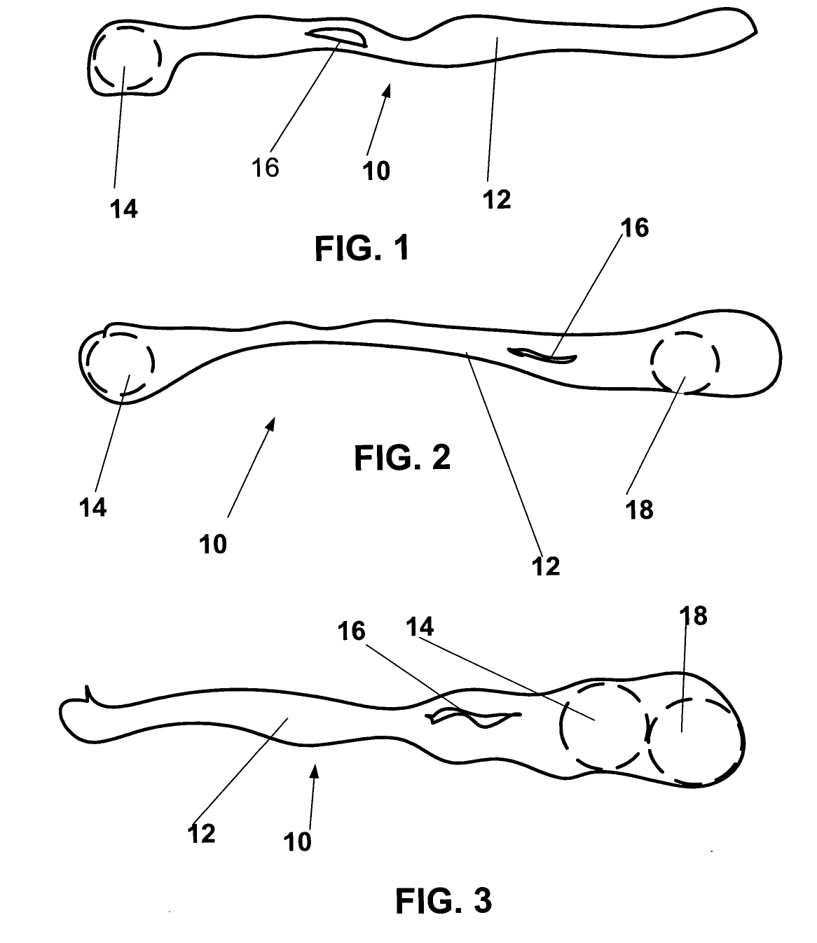

With reference to FIG. 1 of the present invention, a self-acupressure device 10 is exemplified as a hollow elongated flexible casing 12 with an acupressure element 14 inserted into the hollow elongated flexible casing 12 and displaced to one end of the casing. The elongated casing 12 is made from a flexible non-stretchable material such as fabric, e.g., satin, etc.

The casing has an opening 16, preferably in the intermediate position of the elongated casing 12 for inserting or exchanging the acupressure elements into the casing. It is understood that the opening 16 that is made in the form of a slit that should have dimensions allowing insertion of the acupressure element 14 into the casing 12. The slit 16 may extend in the longitudinal direction of the casing 12, though it could be oriented in a different orientation relative to the axis of the elongated casing 12. It is understood that the opening 16 can be closeable, e.g., by buttons, by sewing, or by other means.

FIG. 2 shows the second embodiment of the device 10 where an additional acupressure element 18 can be inserted into the casing 12 through the same opening 16 and positioned at the other end of the casing.

FIG. 3 shows the second embodiment of the device 10 where both acupressure elements 14 and 18 are shifted to one end of the casing 12.

In preferred embodiments, the hollow elongated flexible casing 12 has dimensions of about 90 cm to 120 cm in length, and from 9 cm to 16 in diameter, although larger or smaller sizes are also possible. The opening 16 is closed to ensure only the elements 14 and 18 are allowed in order to avoid any injuries that may be caused by the harder objects which do not belong to the said device.

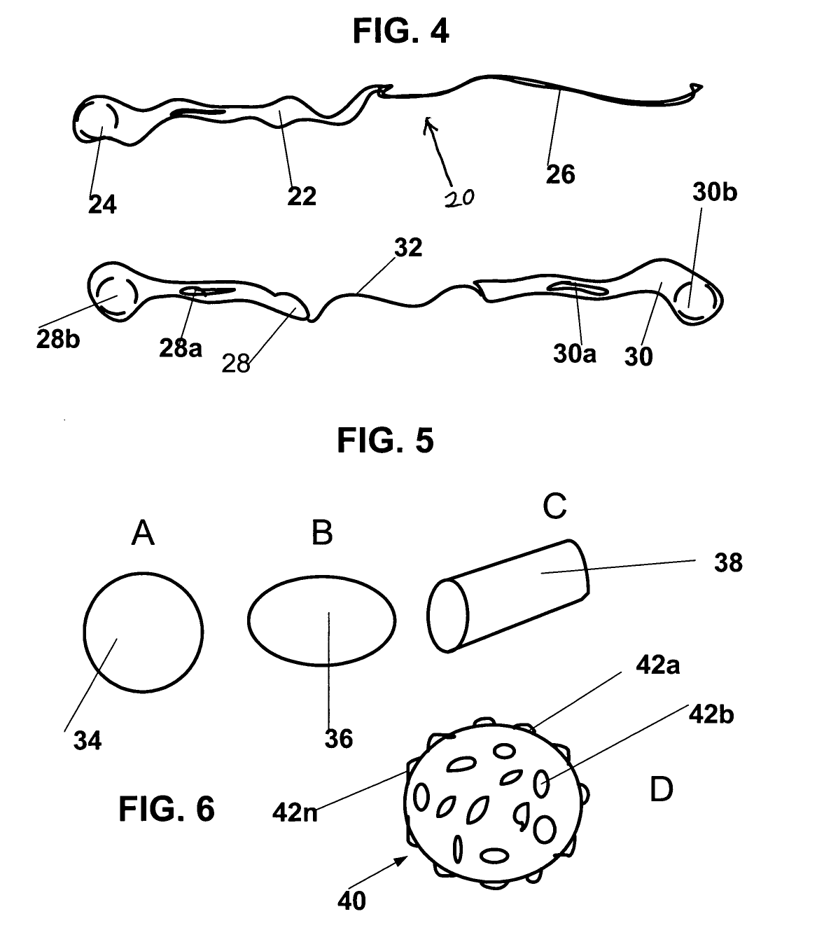

In the embodiment of FIG. 4, a self-acupressure device 20 is provided, comprising at least one casing 22 for containing at least one acupressure element 24, and a cord 26 one end of which is connected to the casing 22. The other end of the cord can be used for holding or wrapping it around the user's hand.

In the embodiment of FIG. 5, two casings 28 and 30 with respective slits 28a and 30a are used and connected to opposite ends of a non-hollow, i.e., string-like flexible element 32, such as a cord, belt, etc., with respective acupressure elements 28b and 30b located in the casings 28 and 30, respectively.

If necessary, the slits 16, 28a, and 30a can be closed by sewing or by using a friction force between tiny hook-and-loop connections of the type produced under the trademark “Velcro”, or by other means.

The cord or the belt 32 can be formed from any suitable material that is sufficiently strong to provide durability to the device. The cord can be constructed from any suitable natural or synthetic material, or combinations thereof, and can be in the form of a thin string or a rope, if desired. The panel or belt likewise can be formed from any suitable material, preferably a woven or nonwoven fabric.

As shown in FIG. 6, the acupressure elements may be made in the form of a resilient ball 34 (FIG. 6A), an oval ball 36 (FIG. 6B), a cylindrical resilient body 38 (FIG. 6C), or a resilient body 40 (FIG. 6D) of any of the aforementioned shapes having a bumpy surface with a plurality of protuberances 42a, 42b, . . . 42n. The acupressure elements 34-40 can be made from a resilient material, such as rubber, or may be made from a non-resilient material, e.g., a casing filled with a bulky material such as grains or seeds.

By selecting the acupressure elements of different hardness and shape, it become possible to imitate the pressure performed by the palm of a human hand by selecting a soft acupressure element and to imitate the pressure performed by the thumb of a human hand by selecting a hard acupressure element.

Methods of Use

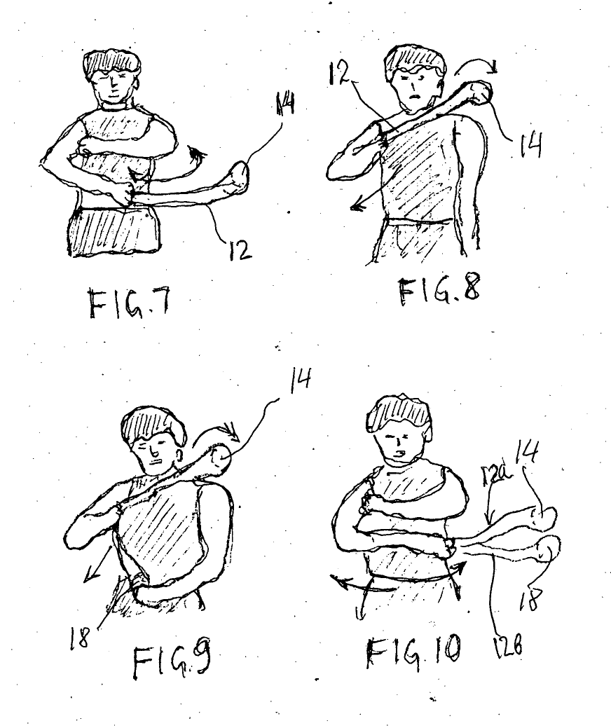

In an additional aspect, a method of self-acupressure conducted by a subject in need thereof is provided. According to one embodiment of the method, the elongated flexible element, such as the hollow casing 12 (FIG. 1) or a cord 26 (FIG. 4) as described above is grasped, and the acupressure element or elements such as the ball 14 (FIG. 1) or balls 14, 18 (FIG. 2) is/are swung to strike the selected areas of his/her body, e.g., the back. The subject can hold the elongated flexible casing 12 or cord 26 at one end thereof and make a good use of acceleration force and swing it horizontally from side to side (as shown in FIG. 7), or diagonally over the shoulder and below the armpit (as shown in FIG. 8) repetitively to strike the selected areas on the subject's body, preferably the back. A subject can swing the acupressure elements 14, 18, 24, 28b (FIGS. 1-4), that may be in any form shown in FIG. 6, as fast or slow, as gentle or hard, as he/she wishes to obtain a desired treatment effect. Furthermore, the subject may adjust the frequency and amplitude of the stimulations by adjusting his/her own swinging motions.

As shown in FIG. 9, a subject may grasp the device at a convenient location by one hand and hold an acupressure element such as the ball 14 by the other hand and swing the other acupressure element 18 to make contact with the selected area on his/her strained back. A subject can also grasp the middle part of the elongated flexible casing 12 (FIG. 10), fold the elongated flexible casing 12 to form two parallel branches 12a, 12b (FIG. 10) with a respective acupressure element in each of the branches, and swing the branches with the acupressure elements 14, 18 side to side in a horizontal plane. The two acupressure elements 14, 18 in two parallel branches may also be swung diagonally in a manner shown in FIG. 8, where only one acupressure element 14 is shown only as an example.

Furthermore, a subject can shift the two acupressure elements 14, 18 into one end of the elongated flexible casing 12 and wrap the empty end of the elongated casing around his/her hand and swing the two acupressure elements at one end of the elongated flexible casing to make contact with the selected areas on his/her strained back in the manner depicted in FIGS. 7 and 8, where one element 14 is shown only as an example.

The above-mentioned methods are not intended only for the back. A skilled user can use the device to provide acupressure to almost any part of the body, e.g., shoulder, chest, hip, thigh, knee, leg, ankle, feet. The subject can also use different motions other than swinging the device, to provide a beneficial contact using the acupressure device.

Thus, it has been shown that the invention provides a self-acupressure device for self-acupressure that is easy to use, readily portable and inexpensive to manufacture, enables the subject to give acupressure to the selected spots, especially on the back, allows acupressure stimulations with the intensity, and frequency as the subject prefers, provides a device for acupressure with little effort by the user in the absence of electrical power, and enables the subject to select and change the intensity and frequency of acupressure stimulations as he/she wishes. The invention also provides a method of self-acupressure treatment that is simple and can be performed in any convenient place without the use of bulky equipment.

Although the invention has been described with reference to specific examples and drawings, it is understood that these examples and drawings should not be construed as limiting the application of the invention and that any changes and modifications are possible without departure from the scope of the attached patent claims. For example, the hollow flexible elongated casing can be made from materials other than those indicated in the specification. Two separate hollow casings with acupressure elements at their ends can be used simultaneously in a manner shown in FIG. 10. Three or more balls of smaller diameters can be used at one or both ends of the hollow casing. The devices can further comprise a grip associated with the hollow casing or the cord, which can be integral to the cord or attached to the cord.

I claim:

1. A self acupressure device comprising at least one acupressure element and at least one hollow elongated flexible casing having two ends for containing at least one acupressure element moveable inside said elongated flexible casing, said at least one acupressure element being positioned at one end of the elongated flexible casing when said self acupressure device is in use.

2. The self acupressure device of claim 1, wherein the hollow flexible casing contains means for replacing or adding acupressure elements.

3. The device of claim 1, further comprising two or more acupressure elements.

4. The device of claim 3, wherein the acupressure elements are located at one end of the hollow elongated flexible casing or are at both ends the hollow elongated flexible casing.

5. The self acupressure device of claim 2, wherein the means for replacing or adding acupressure elements comprises an opening in said hollow elongated flexible casing having dimensions sufficient for inserting said at least one acupressure element.

6. The device of claim 1, wherein the hollow elongated flexible casing is made from a non-stretchable material.

7. The device of claim 1, wherein the acupressure element is made from a material selected from the group consisting of a natural material, synthetic material, semi-synthetic material, and a mixture of the above materials.

8. The device of claim 7, wherein said material of the acupressure element is a material selected from a resilient material and a non-resilient material.

9. The device of claim 1, wherein the acupressure element has a shape selected from a substantially round, oval, or cylindrical.

10. The device of claim 4, wherein the acupressure element is made from a material selected from the group consisting of a natural material, synthetic material, semisynthetic material, and a mixture of the above materials.

11. The device of claim 10, wherein the acupressure element has a shape selected from a substantially round, oval, or cylindrical.

12. The device of claim 1, further comprising a non-hollow string-like flexible element which is connected to said at least one hollow elongated flexible casing.

13. The device of claim 12, where said device further comprising a second hollow elongated flexible element with a second acupressure element and wherein said non-hollow string-like flexible element is located between and connected to both said hollow elongated flexible element and said second hollow elongated flexible element.

14. The self acupressure device of claim 5, wherein said opening is a closable opening.

15. A method of performing a self-acupressure procedure for a subject in need thereof, comprising the steps of: providing at least one acupressure element and an elongated flexible casing having two ends and allowing said acupressure element to be inserted into said elongated flexible element and freely slide in it between said two ends; inserting said at least one acupressure element into said elongated flexible casing; and performing the self-acupressure procedure by striking a selected spot of the subject's body with said acupressure element which freely slides to one of said two ends of the elongated flexible casing.

16. The method of claim 15, wherein said step of striking a selected spot of the subject's body are swinging motions in both directions for alternately striking opposite sides of the subject's body.

17. The method of claim 16, where said flexible elongated element casing is made from a non-stretchable material and where said alternately striking a selected spot of the subject's body is selected from the group of motions consisting of alternating crisscross swinging motions over the shoulder of one arm of said subject and under the armpit of the other arm of said subject and alternating crisscross swinging motions from one side of the subject's body to another side of subject's body in a horizontal plane.

18. The method of claim 17, comprising the steps of holding one end of said elongated flexible casing by one hand, grasping said elongated flexible casing substantially in the middle of the length of said elongated flexible casing by another hand, and performing said alternating striking with said another hand.

19. The method of claim 16, wherein two said acupressure elements are inserted into said flexible elongated casing.

20. The method of claim 17, wherein two acupressure elements are inserted into said flexible elongated casing.

21. The method of claim 20, where said two acupressure elements are positions located in positions selected from the group consisting of two said acupressure elements at one end of said elongated flexible casing and each of said two acupressure elements at each end of said elongated casing.

22. The method of claim 21, further comprising the steps of shifting said acupressure elements to opposite ends of said elongated flexible casing, grasping said elongated flexible casing substantially in the middle of the length thereof by one hand, folding said opposite ends into two parallel branches, and performing said alternating striking with said one hand.

23. The method of claim 17, comprising the step of selecting said acupressure elements from the group consisting of acupressure elements of different hardness and shape for imitating the pressure performed by the palm of a human hand by selecting a soft acupressure element and imitating the pressure performed by the thumb of a human hand by selecting a hard acupressure element.
